(19) United States
(12) Patent Application Publication
(10) Pub. No.: US 2013/0345305 A1
(43) Pub. Date: Dec. 26, 2013

(54) POWDER EMULSIFIER

(71) Applicant: Jan-Olof Lundberg, Malmo (SE)

(72) Inventor: Jan-Olof Lundberg, Malmo (SE)

(73) Assignee: LYCKEBY CULINAR AB, Fjälkinge (SE)

(21) Appl. No.: 13/979,822

(22) PCT Filed: Jan. 13, 2012
(86) PCT No.: PCT/SE2012/050024

§ 371 Date: Sep. 10, 2013

(30) Foreign Application Priority Data
Jan. 21, 2011 (SE) 1150035-2

(51) Int. Cl. A23L 1/052; A61K 31/202; A61K 47/38

(52) U.S. Cl. CPC A23L 1/052; A61K 47/38; A61K 31/202
USPC 514/560; 426/615; 426/637; 426/634; 426/72; 426/602; 426/578; 514/781

(57) ABSTRACT

The present invention relates to an emulsifier comprising vegetable fibrous material with natural or artificial cavities and comprising at least cellulose, hemicellulose, soluble fibrous material and starch. The vegetable fibrous material is based on e.g. potato. The emulsifier also comprises a lipophilic phase constituting a maximum of 75% by weight of the total weight, wherein the emulsifier is in the form of a powder or a paste. The vegetable fibrous material constitutes a support phase in the emulsifier, and the emulsifier also comprises some form of dextrose, dextrose polymer, dextrin or dextrin polymer or a derivate thereof.

### TECHNICAL FIELD OF THE INVENTION

The present invention relates to an emulsifier comprising vegetable fibrous material with natural or artificial cavities.

### BACKGROUND ART

The fact that there are different types of emulsifiers has been known for many years. In the food industry many types of such emulsifiers are used. One example is margarine, which is an emulsion of water in oil. Emulsions can also be found in e.g. milk products and ice cream products. Sausage emulsions is another example.

Furthermore, there are vegetable fibrous materials on the market today. A flavour release material is e.g. described in WO07123466 A1. Flavour release materials comprise a vegetable fibrous material with natural and artificial cavities, and a solution comprising at least one lipophilic flavouring and at least one lipophilic component, the lipophilic solution being applied to the cavities of the fibrous material. The flavour release material is contemplated for bringing about a controlled release of a flavouring in a food matrix, which implies a release of the individual flavour chemicals (flavourings) when release is desired, i.e. not to a greater extent by chemical interactions with other ingredients in the solution or by evaporation when heated during food processes. Controlled release is said to be achieved when the flavour release material according to WO07123466 A1 is exposed to mechanical action, for instance chewing, since the flavourings then migrate from the fat phase into the aqueous phase (saliva) in the oral cavity as the vegetable fibrous material is disintegrated. As is mentioned in WO07123466 A1, the flavour release material is said to have the advantage of being able to accomplish controlled release of flavourings. Since the flavourings are protected in the cavities of the fibers in the material by means of lipophilic components, the migration of the flavourings into the solution is restricted.

An object of the present invention is to provide a new type of emulsifier with improved protective characteristics for e.g. flavourings and other desirable substances.

An object of the present invention is to provide a new type of emulsifier which can be used in aqueous matrices in end products, such as foodstuff, food supplements, chemico-technical products or pharmaceutical preparations, so that lipophilic components may be distributed evenly in hydrophilic matrices. The material aims at functioning as a barrier for mixing in and protect certain components while using the material at a working up application for the production of an end product, but also aims at releasing such components in a controlled way, such as by delayed or sustained release, at the use of the end product. Another purpose of the present invention is to provide different types of applications, such as use of the emulsifier material and various types of products comprising the emulsifier material.

### SUMMARY OF THE INVENTION

The first object given above is achieved using an emulsifier comprising vegetable fibrous material with natural or artificial cavities and which comprises at least cellulose, hemicellulose, soluble fibrous material and starch, wherein the emulsifier also comprises a lipophilic phase constituting a maximum of 75% by weight of the total weight, wherein the emulsifier is in the form of a powder or a paste, wherein the vegetable fibrous material constitutes a support phase in the emulsifier and wherein the emulsifier also comprises some form of dextrose, dextrose polymer, dextrin or dextrine polymer or a derivative thereof.

The emulsifier system according to the present invention functions by creating a food matrix making it possible to distribute lipophilic components in a hydrophilic matrix in an end product (see below), which doesn't necessarily have to be homogenous or as an end product comprise a great amount of water. This is discussed more below.

As is known, an emulsion is a mixture of two liquids that normally don't mix easily, e.g. water and oil or fat. An emulsion is produced by breaking a substance into such small droplets that they float freely in the surrounding liquid, i.e. in the other substance. This can be done in various ways, which results in that there are various types of emulsions, such as emulsions of the type “water in oil” and the type “oil in water”. An emulsifier may thus be defined as a substance that is used to achieve a uniform mixture of a product, such as an emulsion, which consists of two or more components, where several components don't easily mix with each other, such as an oil phase in a water phase according to the above. Further, as an explanation, an emulsifier consists of water soluble hydrophilic components and water-insoluble, oil/fat-soluble lipophilic (hydrophobic) parts. When an emulsifier is added to a mixture of water and oil the emulsifier will be placed at the interface between these so that the hydrophilic part of the emulsifier is anchored in the water phase and the lipophilic part is anchored in the oil/fat-phase.

For the present description it should be mentioned that the expression “powder form or a paste” only includes solid forms of material and forms of a suspension (paste, sol), which means a mixture of solid particles dispersed in a liquid. A material that is totally liquid is, however, not encompassed.

### BRIEF DESCRIPTION OF THE DRAWING

FIG. 1 shows a type of end product in the form of “snacks balls” comprising the powder emulsifier according the present invention.

### SPECIFIC EMBODIMENTS OF THE INVENTION

The emulsifier according to the present invention is foremost applicable for emulsions of the type “oil-in-water”, hereafter called “O/W-emulsions” at some places of the description. In this case oil is dispersed in an aqueous phase. According to a specific embodiment of the present invention the emulsifier therefore is of the type used for “O/W”-emulsions.

An emulsifier is often defined by a so called HLB-value. The HLB-value of an emulsifier can be defined starting from the relationship between the hydrophilic (water attracting) and hydrophobic (water repelling) groups of the emulsifier. The higher the HLB-value the more water soluble the emulsifier is. According to one specific embodiment of the present invention the emulsifier has a HLB-value corresponding to at least 8. According to another specific embodiment the emulsifier has a HLB-value in an interval corresponding to from 12 to 18, such as a value between 12 and 16. HLB-values according to the above are suitable for emulsifiers that are intended for oil-in-water-emulsions.

The fibrous material according to the present invention may be derived from various materials of origin. In principle may many possible starting materials of the fibrous material be used, such as fibrous material based on potato, beetroot, pea, tapioca, carrot or parsnip. Mixtures of these are also possible and also other types of fibrous materials can be used.

Furthermore, some proportions, such as the starch percentage, may vary, e.g. based on the intended application, but it is also possible that this level is enriched in the powder emulsifier. The starch will always be present since it is of importance for the emulgating effect in aqueous phase in end products according to the present invention, on one hand by swelling and on the other hand by pasting. Variants of such end products comprising an emulsifier according to the present invention are discussed more below. Since starch may be present in both bound form and free form and at different proportions, the pasting and thereby the viscosity may be controlled. This in turn controls the distribution rate in an aqueous medium. By controlling the degree of pasting the temperature for release may be controlled. Totally pasted starch gives maximum viscosity at room temperature while unpasted starch gives a low viscosity at room temperature. The homogenity is further controlled via the starch and the viscosity of the hydrophilic phase in the end product (see below), which partly is controlled by the viscosity of the starch in the fibrous raw material.

Also other substances in the fibrous material part of the emulsifier may be enriched or reduced according to the present invention. Furthermore, some substances of interest may occur naturally in the raw material. This appears e.g. below, where some possible substances are brought out, such as dextrose, dextrose polymer, dextrin or dextrin polymer or a derivative thereof.

The emulsifier according to the present invention may be used in many types of applications or products. The products may for instance be foodstuff, food supplements, such as food colouring, chemico-technical products, such as paint and e.g. pharmaceutical preparations. For paints is above all a fat-soluble food colouring of interest, but also regular paint is a possible end product in which the emulsifier may be contained. According to one specific embodiment a product comprising an emulsifier according to the present invention is contemplated, wherein the product is chosen from one of a pharmaceutical preparation, a foodstuff, food supplement or a chemico-technical product and wherein the product further comprises at least one, with regard to the product, active lipophilic component. With active lipophilic component is contemplated, for a food supplement, e.g. different types of vitamins, sweeteners, but also e.g. colouring, such as in a food colouring according to the present invention, for a pharmaceutical preparation an active substance for the contemplated indication, i.e. a pharmaceutical substance, and for a chemico-technical product a substance which is active for the contemplated use, such as a colorant/pigment for a paint substance. Mixtures of a number of so called active components are also possible according to the present invention, i.e. in for example a foodstuff or a food supplement there may be colorants as well as some kind of active pharmaceutical component present.

The present emulsifier has a number of advantages in products according to the above. Firstly, the emulsifier brings about a protective effect, such as against oxidation, since the oil phase and the fibrous matrix protect against air, for one or more active components defined according to the above. The active component/components follow the lipophilic phase for incorporation or encapsulation in the cavities of the fibrous material. This results in that the release of the active component/components may be controlled, such as delayed, prolonged or hindered. Controlled release may refer to the release of flavourings by mechanical action or decomposition of a foodstuff. For example when a foodstuff comprising the emulsifier and the fibrous material is chewed and thereby decomposed, incorporated flavourings may be released. This effect is the same type of effect that is described for the decomposition of the flavour release material according to WO07123466 A1.

Delayed or prolonged release may for instance be of interest at the incorporation of active pharmaceutical substances. It is also possible that such active substances may be incorporated in a food product, such as an emulsion. When eating such a foodstuff it is possible to ensure that a portion of the active substance that is big enough is released in the stomach. Such emulsion pharmaceuticals or other solid types of pharmaceutical products that are not foodstuffs, but are intended for oral consumption, are also possible according to the present invention. By using an emulsifier according to the present invention in such products a similar effect may be achieved as is achieved when gelatine capsules are used today, i.e. a protective effect for an active substance which is to be released in e.g. the stomach. To further strengthen such an effect, and to fully imitate a gelatine capsule intended to comprise pharmaceutical substances, a product according to the present invention may also be coated, for instance with dextrine, dextrine polymer, dextrose or a dextrose polymer.

In addition to giving e.g. “slow release” for a pharmaceutical substance also taste masking can be achieved using an emulsifier according to the present invention. For instance when certain active components, such as pharmaceutical components, have a rank or unpleasant taste the emulsifier according to the present invention may result in that the consumer doesn't feel the taste of these as much, or at all, as the release can take place fully in for instance the stomach. In that way a similar effect can be achieved which otherwise is accomplished in products by coating of the substances the taste of which is to be masked. As an example antibiotics may be mentioned as a suitable substance for masking of the taste by the incorporation in a product comprising an emulsifier according to the present invention. Furthermore, the release may be delayed in comparison to a regular tablet formulation comprising antibiotics today.

Active substances in a pharmaceutical application according to the present invention comprises dietary supplements, such as vitamins, minerals and other wholesome supplements, such as omega 3-fatty acid or omega 6-fatty acid etcetera, but of course also pharmaceutical substances which today are found in pharmaceuticals in capsules or tablets or the like.

In a colour according to the present invention the at least one active component is a colorant. The colour product may be a paint product as well as a food colouring, such as the food colourings described in the FIGURE description below.

In addition to various application types the products may also have various physical nature. According to one embodiment the product is an emulsion of the type oil-in-water. Such an emulsion may for instance be a paint or a foodstuff, but also a pharmaceutical preparation as mentioned above. The emulsions according to the present invention consist of three phases, which phases are water, oil and fibrous material, where the fibrous material is the base in the paste or the powder and is considered a support phase for accomplishing the emulsion. As is realised from above, the end products, for instance a pharmaceutical preparation, foodstuff or colorant, according to the present invention also have a hydrophilic phase. Consequently, it is the emulsifier with its fibrous part (support phase) and lipophilic phase that is added to an aqueous matrix for the production of these end products according to the present invention.

The active components are mixed in the lipophilic phase. The lipophilic phase is protected inside the cavities of the fibrous material. By this protection of active components the emulsifier comprising the fibrous material and its lipophilic phase holding the active components may thereafter be added to an aqueous system, dispersing the lipophilic phase in the water phase, resulting in that the lipophilic part with its active components is protected by the fibrous material and the produced emulsion and thereby is not dissolved out into the aqueous phase. The protection also applies against oxidation, which is mentioned above.

As is mentioned above, different types of physical end products are possible to produce using the emulsifier according to the present invention. This means that not only end products in the form of emulsions are possible. Different types of end products have in common that water is present at some point during the processing. The water content may, however, vary for different applications, both during the processing and in the end product.

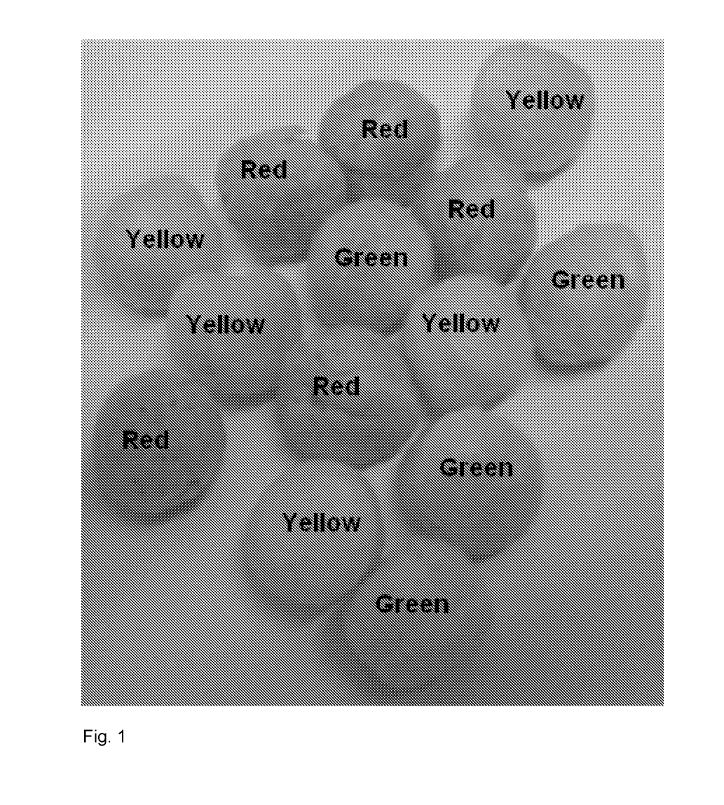

For instance is it possible that the water content is very low in some foodstuff end products according to the present invention, for instance in certain snacks, but where the water content has been higher during the food process for the production of the snacks. When heating by for instance roasting, boiling or drying is present at the production of a foodstuff the water content is lowered. The emulsifier according to the present invention is in these cases especially important during the processing to give the end product the intended taste and/or colour. By introducing the third phase consisting of the fibrous material in an emulsifier according to the present invention the mixture of water/oil is controlled and stabilised before it is further processed. An example of a snacks product where the food colorant has been maintained after extrusion and heating is shown in FIG. 1. Starting from what is described above, according to a specific embodiment of the present invention, the product is a solid product, such as a solid foodstuff, e.g. snacks, such as chips, cheese doodles or the like, or breakfast cereals or similar solid foodstuffs, or products in the form of a paste.

The products according to the present invention may also comprise other substances. According to one embodiment the product therefore also comprises some form of dextrose, dextrose polymer, dextrin or dextrin polymer or a derivative thereof, such as maltodextrin (glucose syrup). These substances or the like may be added, may be present naturally in raw material comprised in the emulsifier according to the present invention or for instance may be enriched through addition. The relationship between starch, fibrous material and for instance dextrose polymer may vary substantially. As an example it can be mentioned that the share of starch plus dextrose polymer to fibrous material may vary between 9:1 and 1:3.

Also described according to the present invention is the use of an emulsifier according to the invention. According to one specific embodiment the use of an emulsifier according to the present invention is contemplated for the creation of an emulsion of the type oil-in-water. As is mentioned above, this use of lipophilic components that are comprised may be evenly dispersed in an aqueous system, i.e. in a hydrophilic matrix.

As is described above, even if an aqueous phase is comprised during the use, the water content may be reduced during the process, for instance by different types of heat treatments or the like. The present invention therefore also contemplates the use of an emulsifier according to the present invention for the production of a solid product or a product in the form of a paste.

As is mentioned above, typical end products, both solid and emulsion based, are e.g. pharmaceutical preparations, foodstuffs, food supplements, such as food colorants, or chemico-technical products, such as paint substances. The products have in common that at least one active component of the product is incorporated in the lipophilic part that is protected inside the fibrous material. In that way the release of such active components is controlled even if the emulsifier is mixed with aqueous phase. The present invention therefore also contemplates use of an emulsifier according to the present invention for the encapsulation of at least one, with reference to the end product, active component.

There are many types of processes for manufacturing that are used today where the emulsifier according to the present invention is applicable. In for instance foodstuff processes where starch is present, pasting, hydrolysation and dextrination are typical examples of such processes. The emulsifier according to the present invention is suitable for all these cases. As is mentioned above, the starch content may vary in the emulsifier and this also controls the degree of dispersibility. This in turn also controls although pasting is suitable to perform for obtaining so called cold water-soluble products. The emulsifier according to the present invention may be used for cold water-soluble products as well as hot water-soluble products.

The products may also be hydrolyzed, for instance by enzymatic hydrolysis or common acid hydrolysis. Furthermore, dextrination, i.e. addition of some kind of dextrose polymer through a coating procedure, is possible with products comprising the emulsifier according to the present invention. As is described above, such a process may be of interest when producing a foodstuff product comprising for instance an active pharmaceutical component or other active component. In that way the delayed release may be strengthened so that in principle all the release of active component takes place in the stomach. In other words may such a product be claimed to imitate a gelatine capsule.

The various process steps described above may be used in series and in various order in for instance a complete foodstuff production. Furthermore is it important to understand that these process steps only are examples, and that also totally different process steps, such as various steps of heat treatment or extrusion, may be included or be used alone with the emulsifier according to the present invention.

### DETAILED DESCRIPTION OF THE FIGURE

FIG. 1 shows “snacks balls” where it is clear that these have different colours. This type of end product according to the invention is an extruded product based on maize flour base that is mixed with about 5% by weight water and about 1% by weight of the emulsifier according to the present invention. To obtain the different colours, different active colour components have been incorporated in the emulsifier to be safely protected in the end product also when water is added. The colour substance for the red end product consists of incorporated carmine, for the yellow it is betacarotene and for the green end product it is chlorophyllin copper. All of these colour substances are heat-sensitive to some extent, and therefore it is important to protect them, such as by use of the emulsifier according to the present invention.

### CONCLUSIONS

The emulsifier according to the present invention has many advantages. The emulsifier is firstly based on totally natural bases of composition, such as vegetable fibrous material and lipophilic phase, e.g. rapeseed oil or other vegetable oils. Furthermore, with the emulsifier according to the present invention it is possible to affect the release of various types of components that are incorporated in the emulsifier in some type of end product. Since the fibrous material incorporates the lipophilic phase, such components may be protected also when aqueous phase is added to a product. As is discussed above, controlled release, such as delayed or prolonged release, may be of interest for pharmaceutical components in pharmaceutical products or foodstuff products with such an effect, and for e.g. colouring agents/pigments in food colouring or other types of paint. Also masking of taste, which also can be said to be a delayed release of certain flavourings, may be achieved in products comprising the emulsifier according to the present invention. Such an effect may also be increased by coating of these end products with some kind of dextrose, dextrose polymer, dextrine or dextrine polymer, such as maltodextrin, as mentioned above.

The emulsifier according to the present invention may be used for many types of processes. In addition to for instance coating proceedings, such as dextrination, some other possible processes that are possible in the foodstuff sector are extrusion, heat treatment and hydrolysation. There are, however, many other variants that also are possible. The core is that the emulsifier may be used when aqueous phase and lipophilic phase are to be mixed in different types of products, especially when some type of active component is to be protected in the product, during the processing thereof or e.g. during storing.

As is mentioned above, several types of products are possible. The common factor is that during at least one point of the processing an emulsion or emulsion like intermediate product has occurred. The water content may on the other hand vary during the processing for different products, especially if you compare possible end products. Emulsion end products, such as sausage emulsion or the like, as well as solid end products, such as chips or other types of snacks, are possible end products comprising the emulsifier according to the present invention. Consequently, it is the ability to keep the fraction of lipophilic components, that are released in an aqueous system, down for the emulsifier according to the present invention that is the common denominator for these different types of end products. In this respect it may be mentioned that a maximum of 5% of the total fat content of the end product may come from the emulsifier. The level of course depends on different factors, such as total content of emulsifier, which normally should be below 5% by weight of the total weight in the end product, together with the type of fibrous material, etcetera. As is evident from the above, the lipophilic phase constitutes a maximum of 75% by weight of the total of the emulsifier. In this respect it may be also be mentioned that a suitable interval for the quantity of the lipophilic phase is from 10 to 75% by weight in the emulsifier to achieve the desired characteristics. Furthermore, it is important to understand that the aqueous matrix of the end product doesn't necessarily have to be fluid as such.

1. An emulsifier comprising vegetable fibrous material with natural or artificial cavities and comprising at least cellulose, hemicellulose, soluble fibrous material and starch, wherein the emulsifier also comprises a lipophilic phase constituting a maximum of 75% by weight of the total weight, wherein the emulsifier is in the form of a powder or a paste, wherein the vegetable fibrous material constitutes a support phase in the emulsifier and wherein the emulsifier also comprises some form of dextrose, dextrose polymer, dextrin or dextrin polymer or a derivative thereof.

2. The emulsifier according to claim 1, wherein the emulsifier is of the type oil-in-water-emulsifier.

3. The emulsifier according to claim 1, wherein the emulsifier has a HLB-value corresponding to at least 8.

4. The emulsifier according to claim 3, wherein the emulsifier has a HLB-value in an interval corresponding to from 12 to 18.

5. The emulsifier according to claim 1, wherein the vegetable fibrous material is based on potato, beetroot, pea, tapioca, carrot or parsnip, or a combination thereof.

6. The emulsifier according to claim 5, wherein the vegetable fibrous material is based on potato raw material.

7. A product comprising an emulsifier according to claim 1, wherein the product is a pharmaceutical preparation, a foodstuff, a food supplement or a chemico-technical product and wherein the product comprises at least one lipophilic component which also is active with regard to the product.

8. A pharmaceutical preparation comprising an emulsifier according to claim 1, wherein the pharmaceutical preparation also comprises at least one active pharmaceutical component.

9. A foodstuff comprising an emulsifier according to claim 1.

10. A foodstuff supplement comprising an emulsifier according to claim 1, wherein the foodstuff supplement also comprises at least one vitamin and/or a sweetener and/or a colorant and/or an active pharmaceutical component.

11. A chemico-technical product comprising an emulsifier according to claim 1.

12. A pharmaceutical preparation according to claim 8, wherein the preparation is an emulsion of the type oil-in-water.

13. A pharmaceutical preparation according to claim 8, wherein the preparation is a solid or a paste.

14. A method comprising using an emulsifier according to claim 1 for creating an emulsion of the type oil-in-water.

15. A method comprising using an emulsifier according to claim 1 for the production of a solid product or a product in the form of a paste.

16. The method according to claim 14, wherein the emulsion or the solid product is a product chosen from a pharmaceutical preparation, a foodstuff, a foodstuff supplement or a chemico-technical product.

17. The method according to claim 14 for the encapsulation of at least one, as regards the end product, active component.
